(19) United States
(12) Patent Application Publication
(10) Pub. No.: US 2009/0306218 A1
(43) Pub. Date: Dec. 10, 2009

(54) Antimicrobial formulations

(76) Inventors: Daniel James Fitzgerald, Leeds (GB); Priscila Gottardello, Bradford (GB)

Correspondence Address: BELL & ASSOCIATES, 58 West Portal Avenue No. 121, SAN FRANCISCO, CA, 94127, US

(21) Appl. No.: 12/311,146

(22) PCT Filed: Sep. 21, 2007
(86) PCT No.: PCT/GB2007/003580

§ 371 Date: Aug. 5, 2009

(30) Foreign Application Priority Data
Sep. 22, 2006 (GB) 0618697.7

(51) Int. Cl. A61K 31/12; A61K 31/05; A61P 31/00; A61P 17/10

(52) U.S. Cl. 514/690; 514/731

(57) ABSTRACT

An antimicrobial formulation containing a synergistic combination of a quinone, such as an optionally substituted benzoquinone or hydroquinone, and a bismuth salt. The formulation may in particular be used against propionibacteria, more particularly to treat skin and skin structure conditions such as acne, or against microbial infections within the oral cavity.

### FIELD OF THE INVENTION

This invention relates to antimicrobial formulations, and to the use of certain combinations of compounds as antimicrobial agents.

### BACKGROUND TO THE INVENTION

Certain quinones, in particular t-butyl hydroquinone (TBHQ), are known for use as antioxidants and in certain contexts as antimicrobial agents.

TBHQ itself for example has been used as a preservative to stabilise foodstuffs, cosmetics and even adhesives. It has also been recognised as an antimycotic (DE-44 34 312).

Other quinones of various types have been disclosed for use as antimicrobial agents—see JP-2003-267910, JP-09-255547, JP-04-211646, JP-04-211644, U.S. Pat. No. 6,228,891, DE-199 11 680 and GB-1,133,897- and as preservatives (JP-02202804 and GB-865,808).

Certain bismuth salts have also been recognised as antibacterial agents, for instance in WO-96/37228 which describes their use in wound treatment compositions together with wound healing agents which can be, inter alia, antioxidants. EP-1 702 621 discloses antimicrobial combinations of thiol-containing complexing agents and bismuth salts—these compositions can be used, for example, as surface disinfectants, topical pharmaceuticals and antimicrobial soaps, such as to treat microbial skin conditions. WO-01/00151 also describes combining a pyrithione with a metal ion source, which can be a bismuth salt, in a topical antimicrobial formulation in particular for antifungal and anti-dandruff use.

In JP-62195329, bismuth salts are also disclosed for use in reducing the side effects of anthracycline-based antibiotics; the bismuth salts are administered orally.

It has now surprisingly been found that when quinones such as TBHQ are combined with bismuth salts, a synergistic effect can be observed on their combined level of antimicrobial activity. As a result, novel antimicrobial formulations can be prepared, in particular for topical application, either with improved efficacy and/or containing lower levels of at least one of the active ingredients than would previously have been thought necessary.

### STATEMENTS OF THE INVENTION

According to a first aspect of the present invention there is provided an antimicrobial formulation containing a quinone and a bismuth salt.

This formulation is preferably suitable for topical application to, and/or contact with, the skin, in particular human skin. The quinone and the bismuth salt are therefore preferably contained in a pharmaceutically acceptable vehicle which can safely be applied to, and/or contacted with, the skin and/or other epithelia. A formulation which is “suitable for” topical application may also be adapted for topical application.

Ideally the formulation is suitable for topical application to areas such as the nares, eyes, scalp or vagina, or to tissue areas within the ears or the oral cavity. Suitability for application to the skin, nares and tissue within the ears and oral cavity is most preferred. In an embodiment, the formulation is suitable for topical application to the skin. In an embodiment, it is suitable for topical application to the nares and tissue within the ears. In an embodiment, it is suitable for topical application to the teeth, gums and/or other areas within the oral cavity.

Suitable vehicles will be well known to those skilled in the art of preparing topical skin care or pharmaceutical preparations. The vehicle will typically be a fluid, which term includes a cream, paste, gel, lotion, foam, ointment or other viscous or semi-viscous fluid, as well as less viscous fluids such as might be used in sprays (for example for nasal use), drops (eg, eye or ear drops), aerosols or mouthwashes. The quinone and the bismuth salt may each independently be present in the form of a solution or suspension, the term “suspension” including emulsions, micellar systems and other multi-phase dispersions.

Either or both of the quinone and the bismuth salt may, whether separately or together, be carried in or on a delivery vehicle which is suitable for targeting or controlling its release at the intended site of administration. Such vehicles include liposomes and other encapsulating entities, for example niosomes, aspasomes, microsponges, microemulsions, hydrogels and solid lipid nanoparticles.

In the context of the present invention, the term “quinone” means cyclohexadiene-1,4-dione (a benzoquinone), or any similar compound containing two or more C═O groups in an unsaturated ring. A quinone may be present in the form of a hydroquinone (hydroxyquinone), in which one or more of the C═O groups is instead present as a C—OH group, or as a radical in which one or more of the C═O groups is present as C—O.. It may be present as a mixture of two or more of these forms, for instance as an equilibrium mixture of a benzoquinone and its corresponding hydroquinone.

The two C═O groups or C—OH groups of a benzo- or hydroquinone may be positioned ortho, meta or para to one another. When positioned ortho to one another, this is known as a cyclohexadiene-1,2-dione or o-benzoquinone or, in the case of the corresponding hydroquinone, a catechol. When positioned meta to one another, this is known as a cyclohexadiene-1,3-dione or an m-benzoquinone or, in the case of the corresponding hydroquinone, a resorcinol. When positioned para to one another, this is known as a cyclohexadiene-1,4-dione or a p-benzoquinone or, in the case of the para-substituted HO-Ph-OH, simply as a p-hydroquinone.

Preferably the two C═O groups or C—OH groups are positioned ortho or para to one another, most preferably para as inp-benzoquinone (cyclohexadiene-1,4-dione) or the corresponding para-substituted hydroquinone HO-Ph-OH.

The quinone may for instance be a benzoquinone (by which is meant an optionally substituted cyclohexadiene dione, typically a cyclohexadiene-1,4-dione or cyclohexadiene-1,2-dione) or its corresponding hydroquinone, by which is meant a compound having an optionally substituted unsaturated 6-membered carbon ring, typically a phenyl ring, substituted with two or more —OH groups. It may be a mixture of a benzoquinone and its corresponding hydroquinone. More preferably the quinone is an optionally substituted hydroquinone. As mentioned above, a hydroquinone may be present in the form of a radical in which one or more of the C—OH groups exists as C—O..

Such compounds may be substituted with one or more groups such as those selected from alkyl groups (in particular C₁ to C₆ or C₁ to C₄ alkyl groups, for instance methyl, ethyl, isopropyl or t-butyl groups); alkoxyl groups (in particular C₁ to C₆ or C₁ to C₄ alkoxyl groups such as methoxyl or ethoxyl); halogens such as fluoro, chloro or bromo, in particular chloro; nitro groups —NO₂; and amino groups —NR₂ (where each R is independently either hydrogen or hydrocarbyl, suitably either hydrogen or C₁ to C₆ alkyl, more suitably either hydrogen or C₁ to C₄ alkyl, for example either hydrogen, methyl or ethyl), in particular NH₂. The quinone may include up to four such substituents, but in particular may be mono- or di-substituted with such groups, more preferably mono-substituted.

In a formulation according to the invention, it may in certain cases be preferred for the quinone not to be an unsubstituted hydroquinone. In some cases it may be preferred for the quinone not to be an unsubstituted benzoquinone.

Preferably the quinone is either an alkyl- or halo-substituted benzoquinone or an alkyl- or halo-substituted hydroquinone, or a mixture of an alkyl- or halo-substituted benzoquinone and its corresponding hydroquinone. More preferably it is an alkyl- or halo-substituted hydroquinone.

Yet more preferably the quinone is either an alkyl-substituted benzoquinone or an alkyl-substituted hydroquinone, or a mixture of an alkyl-substituted benzoquinone and its corresponding hydroquinone. Yet more preferably it is an alkyl-substituted hydroquinone.

A hydroquinone may be substituted with one or more alkyl groups. An alkyl group may be either a straight or a branched chain alkyl group, of which the latter may be preferred, especially where the number of carbon atoms is 3 or greater. It may be or contain cycloalkyl moieties. It may contain for instance from 1 to 12 carbon atoms, preferably from 1 to 10, more preferably from 1 to 8. Particularly preferred alkyl groups are those selected from C₁ to C₆ alkyl groups, more preferably C₁ to C₅ alkyl groups, yet more preferably C₁ to C₄ alkyl groups, for instance methyl, ethyl, iso-propyl or t-butyl groups.

A substituent such as an alkyl or halo group may be attached to a carbon atom of the cyclohexyl ring or to an oxygen atom (thus replacing the hydrogen atom of a hydroxyl group on the cyclohexyl ring). Preferably it is attached to a carbon atom.

The hydroquinone may be substituted with up to six alkyl groups, more preferably up to four alkyl groups, but in particular may be a mono- or di-alkyl hydroquinone, preferably the former.

The hydroquinone may be substituted with one butyl group, which is preferably present at the 2 position; it may however be substituted with more than one butyl group, for instance two or three or four. A butyl group is preferably a t-butyl group.

The hydroquinone may be substituted with two butyl groups, which preferably occupy the 2 and 5 positions. Again the butyl groups are preferably t-butyl groups.

Instead or in addition, the hydroquinone may be substituted with one hexyl group, which is preferably an O-substituted hexyl group replacing the hydrogen atom of a hydroxyl group. The hydroquinone may however be substituted with more than one hexyl group, for instance two or three or even four. A hexyl group may be a straight chain hexyl group.

Instead or in addition, the hydroquinone may be substituted with one methyl group, which is preferably present at the 2 position; it may however be substituted with more than one methyl group, for instance two or three or four or even five. It may for instance be substituted with three methyl groups, which are preferably present at the 2, 3 and 5 positions.

Instead or in addition, the hydroquinone may be substituted with one propyl group, suitably an iso-propyl group, which is preferably present at the 2 position. The hydroquinone may however be substituted with more than one propyl group, for instance two or three or four. A propyl group is again suitably an iso-propyl group.

Instead or in addition, the hydroquinone may be substituted with one ethyl group, which is preferably present at the 2 position. The hydroquinone may however be substituted with more than one ethyl group, for instance two, three, four or even five.

Instead or in addition, the hydroquinone may be substituted with one, two, three or even four pentyl (preferably t-amyl) groups.

In particular the hydroquinone may be substituted with three methyl groups and one hexyl group, the hexyl group preferably replacing the hydrogen atom of a hydroxyl group and the three methyl groups preferably occupying the 2, 3 and 5 positions.

In particular the hydroquinone may be substituted with one methyl and one iso-propyl group, which preferably occupy the 5 and the 2 positions respectively.

In particular the hydroquinone may be substituted with just one t-butyl group, which is preferably present at the 2 position.

The hydroquinone may be substituted with up to six halo groups, more preferably up to four halo groups, but in particular may be a mono- or di-halo hydroquinone, preferably the former. A halo group may be for example either fluoro, chloro, bromo or iodo, suitably either chloro or bromo, more suitably chloro.

In certain cases it may be preferred for the hydroquinone not to be a resorcinol, in particular unsubstituted resorcinol. In some cases it may be preferred for the hydroquinone not to be a catechol, in particular unsubstituted catechol (pyrocatechol).

In some cases it may be preferred for the hydroquinone not to be an alkyl-substituted resorcinol or catechol, in particular an alkyl-substituted resorcinol, more particularly a C₆ to C₉ alkyl-substituted resorcinol, most particularly n-hexylresorcinol.

A substituted hydroquinone may thus be selected from the group consisting of t-butyl hydroquinone (TBHQ); 2,5-di-t-butyl hydroquinone; 2-ethyl-p-hydroquinone; 2-methyl-p-hydroquinone; 2-chloro-p-hydroquinone; and mixtures thereof. It may be selected from the group consisting of TBHQ; 2-ethyl-p-hydroquinone; 2-methyl-p-hydroquinone; 2-chloro-p-hydroquinone; and mixtures thereof. It may in particular be TBHQ, which is a para-hydroquinone substituted at the 2 position with a t-butyl group.

An alkyl-substituted benzoquinone may be substituted with one or more alkyl groups, an alkyl group being as defined above. Such alkyl groups will be attached to carbon atoms of the cyclohexyl ring.

A benzoquinone may be substituted with up to four alkyl groups, but in particular may be a mono- or di-alkyl benzoquinone, preferably the former.

Such a benzoquinone is preferably substituted with one methyl group, which is preferably present at either the 2 or the 5 position; it may be substituted with more than one methyl group, for instance two or three or even four.

Instead or in addition, the benzoquinone is preferably substituted with one propyl group, which is preferably present at the 2 position; it may be substituted with more than one propyl group, for instance two or three or even four. A propyl group is preferably an iso-propyl group.

In particular the benzoquinone may be substituted with one methyl and one iso-propyl group, which preferably occupy the 5 and 2 positions respectively.

The benzoquinone may be substituted with one butyl group (for instance at the 2 position), or with more than one (for instance two, three or four) butyl groups. A butyl group is preferably a t-butyl group.

The benzoquinone may be substituted with two butyl groups, either or preferably both of which is a t-butyl group. These may for instance occupy the 2 and 5 positions, in particular where the benzoquinone is a para-benzoquinone. They may alternatively occupy the 3 and 5 positions, in particular where the benzoquinone is an ortho-benzoquinone.

Instead or in addition, the benzoquinone is preferably substituted with one ethyl group, which is preferably present at the 2 position; it may be substituted with more than one ethyl group, for instance two or three or even four.

Instead or in addition, the benzoquinone may be substituted with one, two, three or even four pentyl (preferably t-amyl) groups.

Instead or in addition, the benzoquinone may be substituted with one, two, three or even four hexyl groups.

The benzoquinone may be substituted with up to four halo groups, but in particular may be a mono- or di-halo benzoquinone, preferably the former. A halo group may be as defined above.

A substituted benzoquinone may thus be selected from the group consisting of thymoquinone (which is a para-benzoquinone substituted at the 2 position with an iso-propyl group and at the 5 position with a methyl group); 2,5-di-t-butyl-1,4-benzoquinone; 2-t-butyl-p-benzoquinone; 2-methyl-p-benzoquinone; 2-chloro-p-benzoquinone; and mixtures thereof. It may be selected from the group consisting of thymoquinone, 2-t-butyl-p-benzoquinone, 2-methyl-p-benzoquinone, 2-chloro-p-benzoquinone and mixtures thereof, or from thymoquinone, 2-t-butyl-p-benzoquinone, 2-chloro-p-benzoquinone and mixtures thereof. More preferably the benzoquinone is thymoquinone or 2-t-butyl-p-benzoquinone, yet more preferably 2-t-butyl-p-benzoquinone.

In the present context a quinone, in particular a hydroquinone or benzoquinone, and more particularly an alkyl-substituted hydroquinone or benzoquinone, may be present in the form of a dimer, oligomer or polymer, the monomer unit of which is a quinone as defined above.

A quinone used in the formulation of the invention, in particular thymoquinone, dithymoquinone or thymohydroquinone, is ideally used in the form of the isolated quinone (whether naturally or synthetically derived, preferably the latter) rather than as part of a plant extract containing a number of different materials.

The quinone may be of the type which is active as an antioxidant.

Most preferred quinones for use according to the invention are those selected from TBHQ, 2-t-butyl-p-benzoquinone (also known simply as t-butyl benzoquinone, or TBBQ) and mixtures thereof.

A formulation according to the invention may contain more than one quinone.

The term “bismuth salt” includes bismuth (III) and (V) salts. Preferably the bismuth salt in a formulation according to the invention is a bismuth (III) salt. The bismuth salt may for instance be selected from bismuth carboxylates, bismuth halides, bismuth sulphadiazine, bismuth sulphate, bismuth nitrate, bismuth subnitrate, bismuth carbonate, bismuth subcarbonate, bismuth oxide, bismuth oxychloride, bismuth hydroxide, bismuth phosphate, bismuth aluminate, bismuth tribromophenate, bismuth thiol, bismuth peptides, bismuth salts of quinolines and their derivatives (eg, bismuth hydroxyquinolines), bismuth pyrithione and other bismuth salts of pyridine thiols, bismuth amino acid salts such as the glycinate, tripotassium dicitrato bismuthate, and mixtures thereof.

Generally speaking the bismuth salt may be either organic or inorganic. It may be a basic bismuth salt (bismuth subsalt) such as the subsalts referred to above.

Suitable bismuth carboxylates include the salicylate, subsalicylate, lactate, citrate, subcitrate, ascorbate, acetate, dipropylacetate, tartrate, sodium tartrate, gluconate, subgallate, benzoate, laurate, myristate, palmitate, propionate, stearate, undecylenate, aspirinate, neodecanoate and ricinoleate. Of these, basic bismuth salicylate (bismuth subsalicylate) and bismuth citrate may be preferred.

Suitable halides include bismuth chloride, bismuth bromide and bismuth iodide.

Preferred bismuth salts may be selected from bismuth halides (in particular bismuth chloride), bismuth nitrates and bismuth carboxylates. More preferred bismuth salts may be selected from bismuth subsalicylate, bismuth salicylate, bismuth subgallate, bismuth subcitrate, bismuth citrate, bismuth acetate, bismuth nitrate and bismuth subnitrate. Yet more preferably the bismuth salt may be selected from bismuth subsalicylate, bismuth citrate and bismuth subnitrate, or from bismuth subsalicylate and bismuth subnitrate, or from bismuth subsalicylate and bismuth citrate. A particularly preferred bismuth salt is bismuth subsalicylate.

In particular where the formulation is for use against propionibacteria and/or for the treatment of acne, it may be preferred for the bismuth salt to be selected from bismuth salicylate, bismuth subsalicylate, bismuth citrate, bismuth subcitrate, colloidal bismuth subcitrate, Ranitidine bismuth citrate, bismuth nitrate, bismuth subnitrate and mixtures thereof. Similar preferences may apply where the formulation is for use against a bacterial infection within the oral cavity.

In some cases it may be preferred for the bismuth salt not to be bismuth (sub)carbonate.

The bismuth salt may be used in an at least partially hydrated form, and may thus be formulated in the presence of an aqueous solvent. Alternatively it may be used in the form of a lipid-soluble salt, suitably in the presence of an organic solvent.

A formulation according to the invention may contain more than one bismuth salt.

In a formulation according to the invention, both the quinone and the bismuth salt are present as active (ie, antimicrobially active) agents. Surprisingly, such agents have been found to act together synergistically to inhibit, and often to prevent, microbial activity. In other words, they have been found to increase one another's activity in a manner which can be synergistic compared to the sum of the activities of the two agents individually.

It is possible that the potentiation of one another's antimicrobial activity by a quinone and a bismuth salt may be at least partly due to the formation of a reaction product having an antimicrobial activity greater than the sum of those of the individual reactants. The invention may thus embrace an antimicrobial formulation containing a reaction product formed between a quinone and a bismuth salt, in particular between TBHQ and a bismuth salt such as bismuth subsalicylate; this reaction product may be formed in situ immediately prior to, or at the point of, use.

In a formulation according to the invention the quinone and the bismuth salt, and their relative proportions, are preferably such as to yield at least an additive level of antimicrobial activity compared to the activities of the individual compounds alone (this is sometimes referred to as an “indifferent” interaction between the compounds). More preferably, the compounds and their relative proportions are such as to yield a synergistic effect on antimicrobial activity, by which is meant that the antimicrobial activity of the combination of the two compounds is greater than the sum of the individual antimicrobial activities of the same amounts of the two compounds used individually. An increased level of activity in these contexts may be manifested by a lower concentration of the compound(s) being needed to inhibit and/or to kill the relevant organism, and/or by a larger zone of inhibition in a disc diffusion assay, and/or by a faster rate of microbial inhibition or killing.

Antimicrobial activity may be growth inhibitory activity or more preferably biocidal (ie, lethal to the relevant organism). Antibacterial activity encompasses activity against both Gram-positive and Gram-negative bacteria. An antimicrobial formulation according to the present invention is preferably at least antibacterially active, more preferably against Gram-positive bacteria. It may be active against bacteria associated with skin or skin-borne infections, yet more preferably against propionibacteria and most preferably against strains of Propionibacterium acnes. It may be active against one or more bacteria associated with infections within the oral cavity. It may be active against one or more anaerobic bacteria.

Thus the formulation may be active against staphylococci (and in cases other Gram-positive cocci such as enterococci and/or streptococci), for example Staphylococcus aureus, and/or against propionibacteria, and/or against bacteria associated with dental or periodontal conditions, for example Streptococcus mutans or in particular Porphyromonas gingivalis. In an embodiment of the invention, the formulation is active against one or more bacteria associated with acne, for example propionibacteria; it may be active against one or more strains of P. acnes and/or in some instances against one or more strains of P. granulosum.

In the context of this invention, activity against a particular species of bacterium may be taken to mean activity against at least one, preferably two or more, strains of that species.

The formulation is preferably active against bacteria, in particular propionibacteria, which are wholly or partially resistant to one or more antibiotics, for instance those which are in common clinical use. It may be active against MRSA bacterial strains, for example. It is ideally active against macrolide-lincosamide-streptogramin (MLS) resistant and/or macrolide-lincosamide-streptogramin-ketolide (MLSK) resistant strains of bacteria. In particular it may be active against erythromycin-resistant, clindamycin-resistant and/or tetracycline-resistant P. acnes strains of bacteria, the term tetracycline here referring to the class of antibiotics including for example minocycline and doxycycline as well as the specific antibiotic known as tetracycline.

Antimicrobial activity may be measured in conventional manner, for instance using the tests described in the examples below. Generally tests for activity involve treating a culture of the relevant micro-organism with the candidate antimicrobial compound, incubating the treated culture under conditions which would ordinarily support growth of the organism, and assessing the level of growth, if any, which can occur in the presence of the candidate compound.

Preferably the quinone used in the present invention has a minimum inhibitory concentration (MIC), at least against propionibacteria, of 200 μg/ml or less, preferably 100 or 70 or 50 or 20 or even 10 μg/ml or less, such as from 0.5 to 50 μg/ml. Its corresponding minimum biocidal concentration (MBC) is preferably 200 μg/ml or less, preferably 150 or 100 or 50 or 20 or even 10 μg/ml or less, such as from 0.5 to 50 μg/ml. Suitably the ratio of its MIC to its MBC is from 0.125 to 1, ideally from 0.5 to 1. More preferably the quinone also exhibits such characteristics in the presence of at least one of, preferably both of, lipid and salt (sodium chloride)—these are species which can be present at the surface of the skin and hence performance in this context can be indicative of suitability for use in topical skin treatment formulations, especially in the context of acne treatment.

Preferably the bismuth salt used in the present invention has an MIC, at least against propionibacteria, of 200 μg/ml or less, preferably 100 or 50 or 40 μg/ml or less or in cases 20 or 10 or even 5 or 2 μg/ml or less, such as from 0.5 to 50 μg/ml. Its corresponding MBC is preferably 200 μg/ml or less, more preferably 100 or 70 μg/ml or less, in cases 50 or 20 or 10 μg/ml or less, such as from 0.5 to 70 or 0.5 to 50 μg/ml. Suitably the ratio of its MIC to its MBC is from 0.125 to 1, ideally from 0.5 to 1. More preferably the bismuth salt also exhibits such characteristics in the presence of at least one of, preferably both of, lipid and sodium chloride.

MIC and MBC values may be measured using conventional assay techniques, for instance as described in the examples below.

The concentration of the quinone in the formulation might suitably be 0.05 or 0.1% w/v or greater, preferably 0.3% w/v or greater. Its concentration might be up to 10% w/v, preferably up to 5% w/v, more preferably up to 3 or 2.5 or 2 or 1% w/v, such as from 0.05 to 5% w/v or from 0.5 to 2.5 or 5% w/v.

The concentration of the bismuth salt in the formulation might suitably be 0.05 or 0.1% w/v or greater, preferably 0.3 or 0.5 or 1% w/v or greater. Its concentration might be up to 5% w/v, preferably up to 3% w/v, more preferably up to 2 or 1% w/v, such as from 0.5 to 5% w/v or from 0.5 to 3% w/v or from 1 to 2% w/v.

Due to the presence of the other compound, it may be possible for the concentration of either the quinone or the bismuth salt, at the site of action when the formulation is applied in vivo, to be less than the MBC, or even than the MIC, of that compound alone. For instance the concentration of at least one of the compounds at this point may be 0.8 or less times its MBC or MIC, such as 0.5 or less, 0.25 or less or 0.125 or less.

Preferably the weight ratio of the quinone in the formulation to that of the bismuth salt is from 500:1 to 1:500, more preferably from 50:1 to 1:50 or from 10:1 to 1:10, yet more preferably from 5:1 to 1:5 or from 5:1 to 1:2 or from 5:1 to 1:1, such as from 2:1 to 1:1.

As described above, the formulation of the invention is preferably suitable for, and more preferably adapted for, topical administration to human or animal, in particular human, skin. It is preferably suitable for, and more preferably adapted for, topical administration to the teeth, gums, skin or other surfaces within the oral cavity. It may also be suitable for, or adapted for, topical administration to other epithelia such as the nares, scalp, ears, eyes or vagina, in particular the nares or ears. It may take the form of a lotion, cream, ointment, foam, paste, gel, suppository or pessary or any other physical form known for topical administration, including for instance a formulation which is, or may be, applied to a carrier such as a sponge, swab, brush, tissue, cloth, wipe, skin patch, dressing or dental fibre to facilitate its topical administration. It may take the form of a nasal spray or of eye or ear drops, or for use within the oral cavity of a toothpaste, mouthwash, dentifrice, lozenge or buccal patch or a formulation carried in or on a dental fibre or tape. It may be intended for pharmaceutical (which includes veterinary but is preferably human) use, for example to treat skin infections or infections within the oral cavity, or as a prophylactic against infections such as MRSA, and/or for cosmetic or other non-medical care purposes (for example, for general hygiene or cleansing).

The vehicle in which the active ingredients are contained may be any vehicle or mixture of vehicles which is suitable for topical application; the type chosen will depend on the intended mode and site of application. Many such vehicles are known to those skilled in the art and are readily available commercially. Examples may for instance be found in Williams' “Transdermal and Topical Drug Delivery”, Pharmaceutical Press, 2003, and other similar reference books. See also Date, A. A. et al, Skin Pharmacol. Physiol., 2006, 19(1): 2-16 for a review of topical drug delivery strategies, and “Oral Hygiene Products and Practice”, 1988, Morton Prader, Ed., Marcel Dekker, Inc., New York, N.Y., USA for information on formulating active substances for delivery within the mouth.

Either or both of the quinone and the bismuth salt may be present in the form of a suspension or other type of multi-phase dispersion, as described above.

Also as described above, the vehicle may be such as to target a desired site and/or time of delivery of the formulation. It may for instance target the formulation to the skin or hair follicles, most preferably to the skin, or to the gums or teeth or other areas within the oral cavity. It may delay or otherwise control release of the formulation over a particular time period. Either or both of the quinone and the bismuth salt may be microencapsulated, for instance in liposomes—particularly suitable liposomes, for topical application to the skin, are those made from stratum corneum lipids, eg, ceramides, fatty acids or cholesterol.

In some cases a polar vehicle may be preferred. Where the formulation is intended for use on the skin, the vehicle may be primarily non-aqueous, although in the case of an anti-acne treatment an aqueous vehicle may be used. The vehicle may be surface-active, in particular when it is intended for use in treating surfaces, for instance to cleanse instruments or working areas. It is suitably volatile. In cases the vehicle may be alcohol-based or silicon-based.

By way of example, a lotion or gel formulation (for instance for application to the skin) may contain a mixture of water, an alcohol such as ethanol or phenoxyethanol and optionally a glycol such as propylene glycol.

A formulation according to the invention may contain standard excipients and/or other additives known for use in pharmaceutical or veterinary formulations, in particular topical skin care formulations. Examples include emollients, perfumes, antioxidants, preservatives, stabilisers, gelling agents and surfactants; others may be found in Williams' “Transdermal and Topical Drug Delivery”, supra.

For the treatment of acne, however, it may be preferred for the formulation not to contain an emollient.

Where a formulation according to the invention is for use inside the mouth, an orally acceptable and systemically non-toxic vehicle will be necessary. For example, where the formulation takes the form of a toothpaste, a typical vehicle might include water and a humectant to provide a liquid base, together with one or more of a thickener, a surfactant and a polishing agent. Suitable humectants include glycerol, sorbitol and polyethylene glycol, and in particular mixtures thereof. A polyethylene glycol humectant may for example have a molecular weight range of from 200 to 1000 or from 400 to 800, such as about 600.

Suitable thickeners for use in toothpaste formulations include natural and synthetic gums and colloids such as carrageenan, xanthan gum and sodium carboxymethyl cellulose, as well as gum tragacanth, starch, polyvinyl pyrrolidone, hydroxyethyl propyl cellulose, hydroxybutyl methyl cellulose, hydroxypropyl methyl cellulose and hydroxyethyl cellulose. Suitable inorganic thickeners include colloidal silica and synthetic hectorite. Mixtures of thickeners may also be used.

Suitable surfactants for use in toothpaste formulations according to the invention include water soluble detergents. In general they may be anionic, nonionic or ampholytic, but are preferably anionic. Examples of suitable anionic surfactants include higher alkyl sulphates such as sodium lauryl sulphate, and higher fatty acid esters of 1,2 dihydroxy propane sulphonate. Examples of suitable water soluble nonionic surfactants include the polymeric condensation products of hydrophilic alkylene oxide group-containing compounds (typically ethylene oxide) with organic hydrophobic compounds (for example those having aliphatic chains of about 12 to 20 carbon atoms). Such products include the “ethoxamers” and include for example the condensation products of poly(ethylene oxide) with fatty acids, fatty alcohols, fatty amides and other fatty moieties, as well as with propylene oxide and polypropylene oxides (the latter being available, for example, under the trade name Pluronic®).

Polishing agents which may be incorporated in a toothpaste formulation according to the invention include siliceous materials, such as precipitated amorphous hydrated silicas, and also sodium bicarbonate, sodium metaphosphate, potassium metaphosphate, tricalcium phosphate, calcium phosphate dihydrate, anhydrous dicalcium phosphate, calcium pyrophosphate, magnesium orthophosphate, trimagnesium phosphate, calcium carbonate, alumina trihydrate, aluminium silicate, zirconlure silicate, calcined alumina, bentonite, silica gel and colloidal silica, and complex amorphous alkali metal aluminosilicates. Mixtures of such polishing agents may also be used.

Where a formulation according to the invention takes the form of a mouthwash or dentifrice, it may for example contain a water/alcohol (eg, water/ethyl alcohol) solution and optionally one or more other ingredients selected for example from flavourings, sweeteners, humectants, surfactants and mixtures thereof. Suitable humectants include those described above, in particular glycerol and sorbitol. One or more additional antibacterial agents may also be included.

Non-soap surfactants (for example nonionic, cationic or amphoteric surfactants) may be preferred for use in mouthwash formulations. Suitable nonionic surfactants include the condensation products of hydrophilic alkylene oxide group-containing compounds with organic hydrophobic compounds, as described above. Other suitable nonionic synthetic detergents include: the polyethylene oxide condensates of alkyl phenols; those derived from the condensation of ethylene oxide with the product resulting from the reaction of propylene oxide and ethylene diamine; the condensation products of aliphatic alcohols having from 8 to 18 carbon atoms with ethylene oxide; and the polyoxyethylene derivatives of fatty acid partial esters of sorbitol anhydride (for example the commercially available Tween® products).

Suitable cationic detergents include quaternary ammonium compounds, in particular those having one long alkyl chain of about 8 to 18 carbon atoms, for example lauryl trimethylammonium chloride, cetyl pyridinium chloride, cetyl trimethylammonium bromide, di-isobutylphenoxyethyldimethylbenzylammonium chloride, coconutalkyltrimethylammonium nitrite, cetyl pyridinium fluoride and the like.

Suitable amphoteric detergents include derivatives of aliphatic secondary and tertiary amines in which one of the aliphatic substituents contains from about 8 to 18 carbon atoms and one contains an anionic water solubilising group such as carboxylate, sulphate, sulphonate, phosphate or phosphonate.

Other suitable surfactants, for use in formulations according to the invention, may be found in McCutcheon's Detergents and Emulsifiers.

An antimicrobial formulation according to the invention may further contain additional active agents. For example, it may contain one or more additional agents selected from anti-acne agents, keratolytics, comedolytics, agents capable of normalising keratinocyte and/or sebocyte function, anti-inflammatories, anti-proliferatives, antibiotics, anti-androgens, sebostatic/sebosuppressive agents, anti-pruritics, immunomodulators, agents which promote wound healing, additional antimicrobial (in particular antibacterial) agents and mixtures thereof. It may in particular contain one or more agents selected from anti-acne agents, keratolytics, comedolytics, sebostatic/sebosuppressive agents, anti-inflammatories and additional antibacterial agents. It may instead or in addition contain one or more agents selected from sunscreens, moisturisers and mixtures thereof.

It may, in particular when used to treat conditions in the mouth, contain one or more agents selected from abrasives, bleaching agents, tooth whitening agents (for example peroxides or sodium perborate), surface active agents/detergents as described above, foaming agents, sources of fluoride ions or fluorine-containing ions, zinc salts, non-cariogenic sweeteners such as saccharin or aspartame, other flavourings such as peppermint or spearmint or aniseed, menthol, desensitising agents, anti-tartar/sequestering agents or anti-calculus agents (for example zinc chloride, zinc acetate or zinc oxide), sodium bicarbonate, enzymes such as lactoperoxidases, humectants as described above, pH regulating buffers, preservatives, colours/dyes (for example chlorophyll or titanium dioxide), plant extracts, anti-plaque agents, additional antibacterial agents, and mixtures thereof.

Suitable additional antibacterial agents, for oral health care formulations, include chlorhexidene, cetyl pyridinium chloride and natural extracts such as sanguinaria extract.

Suitable sources of fluoride or fluorine-containing ions are water soluble fluorides such as water soluble alkali metal fluorides, for example sodium and potassium fluorides; copper fluorides, such as cuprous fluoride; ammonium fluorosilicate; sodium and ammonium fluorozirconates; sodium and potassium monofluorophosphates; and mono-, di- and tri-aluminium fluorophosphates.

Generally speaking a formulation according to the invention may contain one or more agents which enhance the activity of another active agent present in the formulation, or reduce a side effect of such an active, or improve patient compliance on administration of the formulation.

In general an additional antimicrobial agent may for example be selected from the group consisting of biocides, disinfectants, antiseptics, antibiotics, bacteriophages, enzymes, anti-adhesins, immunoglobulins and mixtures thereof; it is preferably active as a bactericide, in particular against propionibacteria and/or staphylococci and/or one or more bacteria associated with oral health problems.

It may however be preferred for the quinone and the bismuth salt to be the only active agents in the formulation, or at least to be the only antimicrobially or antibacterially active agents and/or the only anti-acne active agents.

In cases it may be preferred for a formulation according to the invention not to contain an antibiotic, in particular an anthracycline-based antibiotic for example as referred to in JP-62195329.

In cases it may be preferred for a formulation according to the invention not to contain Vitamin A, beta-carotene, Vitamin E and/or Vitamin E acetate. In cases it may be preferred for the formulation not to contain any of the antioxidants listed at page 20 lines 7-25 of WO-96/37228.

In cases it may be preferred for a formulation according to the invention not to contain a complexing agent, in particular a thiol-containing complexing agent such as those referred to in EP-1 702 621.

A formulation according to the invention may be incorporated into, and hence applied in the form of, another product such as a cosmetic; a skin care preparation; an oral health care product such as a toothpaste, mouthwash or dental floss; a pharmaceutical (which includes veterinary) or cosmeceutical preparation or a toiletry product (for instance a bath or shower additive or a cleansing preparation).

The formulation may be incorporated into another product as a preservative; it may for example be included in a food or beverage, a pharmaceutical preparation, a cosmetic or toiletry product, or a tissue, serum or other body sample, so as to inhibit or prevent microbial activity or growth in the product.

The invention provides, according to a second aspect, a product which incorporates an antimicrobial formulation according to the first aspect of the invention.

The formulation of the invention may be prepared in situ, at or immediately before its point of use, for instance its application to the skin or another surface. Thus according to a third aspect, the present invention provides a kit for preparing an antimicrobial formulation according to the first aspect, the kit comprising a source of a quinone and a source of a bismuth salt, together with instructions for combining the two compounds so as to make the formulation at or before the point of intended use, and/or for the co-administration of the two compounds, for instance to a surface such as the skin. The two compounds may each be present in a suitable respective vehicle.

According to one embodiment, the formulation or kit of the invention may contain both a quinone and a bismuth salt, each encapsulated (for instance microencapsulated) in a separate delivery vehicle; this might for instance allow their release, and hence their contact with one another, only at the intended site of administration.

A fourth aspect of the invention provides a method for preparing an antimicrobial formulation, which method involves mixing together a quinone and a bismuth salt, preferably together with a pharmaceutically acceptable vehicle.

According to a fifth aspect of the invention there is provided a formulation (preferably a formulation according to the first aspect) containing a quinone and a bismuth salt, for use in the treatment of a condition which is caused by, transmitted by and/or exacerbated by (in particular either caused by or transmitted by) microbial, especially bacterial, activity. Such a condition may be for example a skin, skin structure, oral, ocular, aural, nasal or vaginal condition.

According to an embodiment of this fifth aspect of the invention, the formulation is for use in the treatment of a skin or skin structure condition. It may in particular be for use in the treatment of acne (ie, as an anti-acne agent).

In the context of the present invention, treatment of a condition encompasses both therapeutic and prophylactic treatment, of either an infectious or a non-infectious condition, in either a human or animal but in particular a human. It may involve complete or partial eradication of the condition, removal or amelioration of associated symptoms, arresting or slowing subsequent development of the condition, and/or prevention of, or reduction of risk of, subsequent occurrence of the condition. It will typically involve use of the formulation as a microbiocide, in particular as a bactericide and/or bacteriostatic agent, more particularly against propionibacteria and/or Gram-positive cocci such as staphylococci or streptococci and/or bacteria associated with an infection within the oral cavity. Most particularly it may involve use of the formulation against propionibacteria and/or one or more bacteria associated with an infection within the oral cavity, or at least the former.

Treatment may involve the prophylactic treatment of any area of the body, in particular the skin or nares or another epithelial or mucosal surface, against micro-organism infections, including against staphylococcal infections such as those associated with MRSA.

Skin and skin structure conditions which might be treated according to the invention include acne, eczema, superficial infected traumatic lesions, wounds, burns, ulcers, folliculitis, mycoses and other primary and secondary skin and skin structure infections. In particular the formulation may be for use in treating acne or acne lesions (for instance, to reduce acne-related scarring).

Acne is a multifactorial disease of the pilosebaceous follicles of the face and upper trunk, characterised by a variety of inflamed and non-inflamed lesions such as papules, pustules, nodules and open and closed comedones. Its treatment can therefore encompass the treatment (which embraces prevention or reduction) of any of these symptoms, and references to use as an anti-acne agent may be construed accordingly.

The treatment of acne also encompasses the treatment and/or prevention of lesions and/or scarring associated with acne.

In general, the present invention will be used for the treatment of symptoms which are directly due to acne rather than for instance infections which may arise as a consequence of treating acne with other actives such as antibiotics, and/or secondary infections caused by opportunistic pathogens, which can arise in skin already affected by acne.

In another embodiment of the invention, the formulation may be for use as a therapeutic or prophylactic treatment against staphylococci on the skin, or in the nares or ears, which might otherwise cause for example MRSA-associated infections.

The formulation may instead or in addition be used for the therapeutic or prophylactic treatment of a condition within the oral cavity which is caused by, transmitted by and/or exacerbated by microbial, in particular bacterial or fungal and more particularly bacterial, activity. Examples include dental caries, halitosis, oral thrush and periodontal diseases such as gingivitis and periodontitis. The formulation may be used for the creation and/or maintenance of fresh-smelling breath. It may be used for the prevention and/or reduction of plaque formation.

According to the fifth aspect of the invention, the formulation of quinone and bismuth salt may be prepared in situ, at or immediately before the point of administration. This aspect of the invention thus pertains to any use of a quinone and a bismuth salt in the treatment of a microbial (especially bacterial) condition, the two compounds being administered either simultaneously or sequentially.

According to a sixth aspect, the invention provides the use of a quinone and a bismuth salt in the manufacture of a medicament (typically a formulation) for the treatment of a condition which is caused by, transmitted by and/or exacerbated by (in particular either caused by or transmitted by) microbial, especially bacterial, activity. As discussed above, each of the compounds individually or together may be used as an antimicrobial, in particular antibacterial, agent in the medicament, and/or as an anti-acne active agent.

Again the condition may be a skin or skin structure condition such as acne. It may be a microbial infection within the oral cavity, such as those listed above. It may be a condition which is caused by, transmitted by and/or exacerbated by Gram-positive cocci such as staphylococci.

The invention further provides, according to a seventh aspect, the use together of a quinone and a bismuth salt, as an antimicrobial agent, in particular as an antibacterial agent and more particularly as an anti-acne agent, or in the manufacture of an antimicrobial or anti-acne formulation.

An eighth aspect provides a method for controlling the growth of a micro-organism, in particular a bacterium such as a propionibacterium or staphylococcus, the method comprising applying, to an area or surface which is infected or suspected to be infected or capable of becoming infected with the organism, a combination of a quinone and a bismuth salt. Again the two compounds may be applied simultaneously or sequentially.

In this context, “controlling the growth” of a micro-organism embraces inhibiting or preventing its growth, whether completely or partially, as well as killing or inactivating either completely or partially a culture of the organism. It also embraces reducing the risk of subsequent growth of the organism in or on the area or surface being treated. The method of the invention may thus be used to treat an existing occurrence of the organism or to prevent, or reduce the risk of, a potential subsequent occurrence.

Again the area or surface to which the quinone and the bismuth salt are applied will typically be a living surface such as human tissue, in particular the skin. In this case the quinone and the bismuth salt may be applied for therapeutic purposes or for non-therapeutic (eg, purely cosmetic) purposes. Thus the method of the eighth aspect of the invention encompasses a method of treatment of a human or animal patient suffering from or at risk of suffering from a condition which is caused by, transmitted by and/or exacerbated by (in particular either caused by or transmitted by) microbial, especially bacterial, activity, the method involving administering to the patient a therapeutically or prophylactically effective amount of an antimicrobial formulation containing a quinone and a bismuth salt.

Alternatively the active ingredients may be applied to a nonliving surface such as in a hospital or food preparation area. For example the method of the eighth aspect of the invention may be used to treat work surfaces, surgical or other instruments, surgical implants or prostheses, contact lenses, foods, crops, industrial plant, floors and walls (both internal and external), clothing, bedding and many other surfaces.

The method of the eighth aspect of the invention preferably involves applying a formulation according to the first aspect.

A ninth aspect of the invention further provides a method for controlling the growth of a micro-organism, in a product which contains or is suspected to contain or is capable of containing the micro-organism, the method comprising incorporating into the product a combination of a quinone and a bismuth salt.

A tenth aspect of the invention provides the use of a quinone in an antimicrobial or anti-acne formulation, in combination with a bismuth salt, for the purpose of increasing the antimicrobial (in particular antibacterial) and/or anti-acne activity of the formulation and/or of reducing the amount of the bismuth salt in the formulation without undue loss of antimicrobial (in particular antibacterial) or anti-acne activity.

An increase in antimicrobial or anti-acne activity may be as compared to that of the bismuth salt alone, at the same concentration as used when combined with the quinone. Ideally the increase is as compared to the sum of the activities of the quinone and the bismuth salt individually, again at the same respective concentrations as used when the two are combined.

A reduction in the amount of the bismuth salt in the formulation may be as compared to the amount which would otherwise have been used in the formulation in order to achieve a desired level of activity, in particular in order to have acceptable efficacy in the context of its intended use. The reduction may be manifested by reduced side effects which would otherwise have been observed during use of the formulation, for example local irritation and/or undesirable systemic absorption of the bismuth salt. According to the invention, the quinone may therefore be used for the dual purposes of reducing an undesired property of a formulation containing a bismuth salt, without undue loss of antimicrobial or anti-acne activity.

Preferably the quinone is used without any reduction in antimicrobial or anti-acne activity compared to the level exhibited by the formulation prior to addition of the quinone. More preferably it is used to give an increase in antimicrobial or anti-acne activity. It may however be used to reduce the amount of the bismuth salt present, and/or its associated side effects, whilst maintaining the antimicrobial or anti-acne activity of the resultant formulation at a level, albeit lower than that which it would otherwise have exhibited, which is still acceptable in the context of its intended use.

An eleventh aspect of the invention provides the use of a bismuth salt in an antimicrobial or anti-acne formulation, in combination with a quinone, for the purpose of increasing the antimicrobial (in particular antibacterial) and/or anti-acne activity of the formulation and/or of reducing the amount of the quinone in the formulation without undue loss of antimicrobial or anti-acne activity.

An increase in antimicrobial or anti-acne activity may be as compared to that of the quinone alone, at the same concentration as used when combined with the bismuth salt. Ideally the increase is as compared to the sum of the activities of the bismuth salt and quinone individually, again at the same respective concentrations as used when the two are combined.

A reduction in the amount of the quinone in the formulation may be as compared to the amount which would otherwise have been used in the formulation in order to achieve a desired level of activity, in particular in order to have acceptable efficacy in the context of its intended use. The reduction may be manifested by reduced side effects which would otherwise have been observed during use of the formulation, for example local irritation and/or undesirable systemic absorption of the quinone. According to the invention, the bismuth salt may therefore be used for the dual purposes of reducing an undesired property of a formulation containing a quinone, without undue loss of antimicrobial or anti-acne activity.

Preferably the bismuth salt is used without any reduction in antimicrobial or anti-acne activity compared to the level exhibited by the formulation prior to addition of the bismuth salt. More preferably it is used to give an increase in antimicrobial or anti-acne activity. It may however be used to reduce the amount of the quinone present, and/or its associated side effects, whilst maintaining the antimicrobial or anti-acne activity of the resultant formulation at a level, albeit lower than that which it would otherwise have exhibited, which is still acceptable in the context of its intended use.

Throughout the description and claims of this specification, the words “comprise” and “contain” and variations of the words, for example “comprising” and “comprises”, mean “including but not limited to”, and do not exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Preferred features of each aspect of the invention may be as described in connection with any of the other aspects.

Other features of the present invention will become apparent from the following examples. Generally speaking the invention extends to any novel one, or any novel combination, of the features disclosed in this specification (including any accompanying claims and drawings). Thus features, integers, characteristics, compounds, chemical moieties or groups described in conjunction with a particular aspect, embodiment or example of the invention are to be understood to be applicable to any other aspect, embodiment or example described herein unless incompatible therewith.

Moreover unless stated otherwise, any feature disclosed herein may be replaced by an alternative feature serving the same or a similar purpose.

The present invention will now be further described with reference to the following non-limiting examples and the accompanying figures, of which:

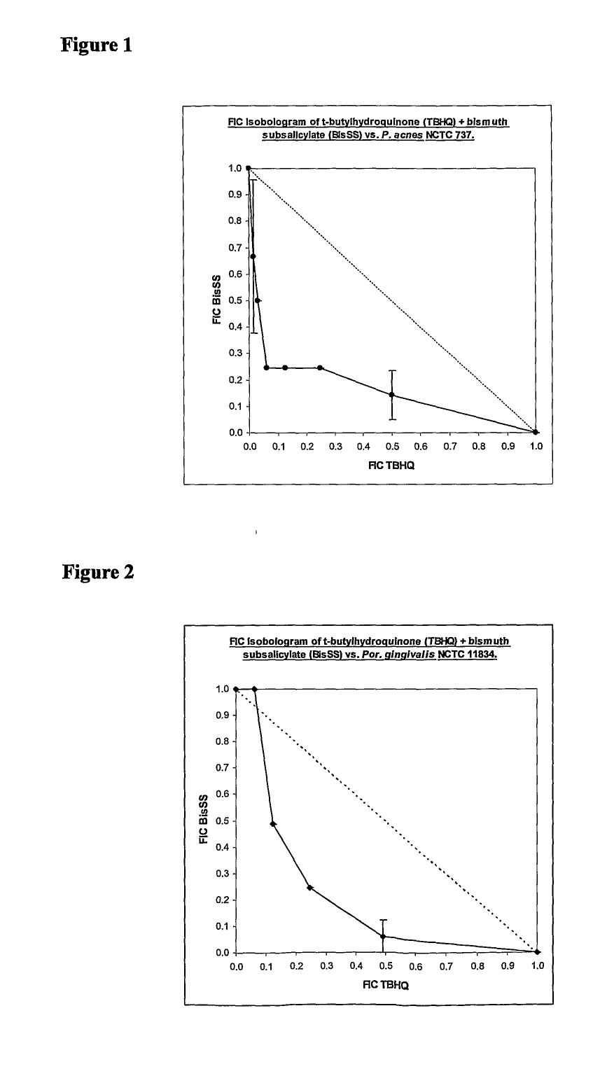

FIG. 1 is an isobologram showing FIC (fractional inhibitory concentration) values for mixtures of TBHQ and bismuth subsalicylate against a propionibacterial strain, as referred to in Example 2 below; and

FIG. 2 is an isobologram showing FIC (fractional inhibitory concentration) values for mixtures of TBHQ and bismuth subsalicylate against a bacterium of the Porphyromonas genus, as referred to in Example 7 below.

### DETAILED DESCRIPTION

Experimental tests were conducted to determine the antimicrobial activities of formulations according to the invention. As a comparison, the antimicrobial activities of formulations containing a quinone or a bismuth salt alone were also measured.

Test Micro-Organisms

The principal test micro-organism used was Propionibacterium acnes NCTC 737. Other propionibacterial strains, including some P. granulosum strains and including some having antibiotic resistance, were also used as test organisms in Example 4. Propionibacteria are known to be involved in acne, which is a complex, multi-factorial skin disease in which P. acnes and other Propionibacterium spp. play key roles. Activity observed against the chosen test organisms is therefore expected to be a reasonable qualitative predictor of activity against micro-organisms responsible for skin and skin structure infections, in particular acne.

All the propionibacteria were cultured and maintained on Wilkins-Chalgren Anaerobe Medium (agar and broth) at pH 6.0; all cultures were incubated anaerobically at 37° C. for 72 hours.

Also tested was Porphyromonas gingivalis NCTC 11834—this is a black pigmented gram-negative anaerobic bacterium belonging to the genus Porphyromonas. Porphyromonas are a human commensal bacterium, predominantly associated with the oral cavity. Clinically, Por. gingivalis is associated with periodontal lesions, infections and adult periodontal disease. Gingivitis (inflammation of the gums that causes bleeding and exposes the base of the teeth) can be a precursor to periodontal disease by allowing Por. gingivalis to infect the areas near the roots of the teeth and thus cause tooth decay and infection.

Activity observed against this micro-organism is expected to be a reasonable qualitative predictor of antimicrobial activity, in particular against micro-organisms responsible for periodontal lesions, infections, and periodontal disease.

Por. gingivalis was cultured and maintained on Wilkins-Chalgren Anaerobe Medium (agar and broth) at pH 7.0; all cultures were incubated anaerobically at 37° C. for 5-7 days.

The following tests were carried out to assess antimicrobial activity against the test organisms.

(a) Minimum Inhibitory Concentration (MIC) Assay

This is a standard international method for quantitatively assessing the antimicrobial activity of a compound in a liquid medium. The method used a sterile 96-well microtitre plate, capable of holding about 200 μl of liquid per well. The wells contained liquid culture medium and ranges of decreasing concentrations of the relevant test compound in doubling dilutions (e.g. 1000, 500, 250, 125 . . . μg/ml, etc. down to 0.49 μg/ml). The culture medium was as described above.

The wells were inoculated with a liquid suspension of freshly grown micro-organism and incubated under the conditions described above. After incubation, the microtitre plate was examined visually (with the aid of a light box) for cloudiness in each well, which would indicate microbial growth. The MIC value was recorded as the lowest concentration of test compound required to inhibit microbial growth, i.e., the lowest concentration for which the liquid in the well remained clear.

The assays were conducted in duplicate (minimum) and included both negative (culture medium with no micro-organisms) and positive (culture medium plus diluting solvent plus micro-organism) controls.

Since inhibition does not necessarily indicate killing of microbial cells, merely that growth as visible to the naked eye has been inhibited, it is desirable to conduct a further test (the MBC assay described below) to establish the concentration of the test compound needed to kill the test organism.

(b) Minimum Bactericidal Concentration (MBC) Assay

This assay, normally carried out after an MIC assay, determines the minimum concentration of a compound that is lethal to the micro-organism being tested.

Following an MIC assay, a 5 μl sample was withdrawn from the first microtitre well that showed positive growth and from all the subsequent wells that showed no growth. These samples were then individually sub-cultured on antibiotic-free agar medium, under the incubation conditions described above. Following incubation they were examined visually for microbial growth. The MBC was taken to be the lowest test compound concentration for which the incubated sample showed no growth.

The ratio of MIC to MBC should ideally be as close to 1 as possible. This facilitates selection of the lowest possible effective concentration of a test compound with a reduced risk of selecting a sub-lethal concentration which could promote resistance or allow the target microbial population to recover.

(c) Agar Dilution MIC Assay

This is a standard international method for quantitatively assessing the antimicrobial activity of a compound in a solid medium. The test compound was prepared to 40× the highest concentration required (e.g., 10 mg/ml for a final concentration of 250 μg/ml) and a series of doubling dilutions were performed in a suitable solvent. A set amount of these antimicrobial stock solutions was then added to molten agar medium (ca. 55° C.), mixed thoroughly, poured into sterile Petri dishes and allowed to cool/set. The culture medium was as described above.

A Multipoint™ Inoculator (AQS Manufacturing Ltd, UK) was used to inoculate the plates by spotting the inocula onto the surface of the agar, delivering approximately 1 to 2 μl per spot (yielding 10⁵ CFU (colony forming units) per spot).

The plate(s) were then incubated under the conditions described above, following which they were examined visually for signs of bacterial growth. The MIC value was ascertained when there was a marked reduction in, or total loss of, growth on the test plate at the lowest concentration as compared to that of the growth on the control plate.

The assays were conducted in duplicate and included a positive control (culture medium, diluting solvent and inoculum).

(d) Disc Diffusion Assay (DDA)

This is an internationally recognised standard method for qualitatively assessing the antimicrobial activity of a compound.

A sterile paper disc was impregnated with a sample of the test compound in a suitable solvent and 30 minutes allowed for the solvents to evaporate (where possible). The disc was then placed on an agar plate onto which the test micro-organism had been inoculated. The plate was then incubated under the conditions described above, following which it was examined visually for signs of microbial growth. If the test compound had antimicrobial activity, a circular zone of no growth would be obtained around the disc. The diameter of this zone of “inhibition” was measured using a ProtoCOL™ automated zone sizer (Synbiosis, Cambridge, UK). In general, a greater diameter and/or area of the zone of inhibition indicates a greater antimicrobial activity in the relevant test compound, although other factors such as test compound mobility through the agar gel may also influence the result.

(e) Synergy Disc Diffusion Assay (SDDA)

This is a variation on the DDA method, in which two compounds are tested together for their combined antimicrobial activity.

Two test compounds A and B were placed on a single paper disc and the above described DDA procedure repeated. An increase in diameter of the zone of inhibition, compared to the greater of the zone diameters for the two compounds individually, was taken to indicate potential antimicrobial synergy. In practical terms, an increase of greater than 5 mm could be treated as significant.

(f) Fractional Inhibitory Concentration (FIC) Assay

This assay was used to determine the mode of interaction between two antimicrobial test compounds A and B. It was similar to the MIC assay, utilising a 96-well microtitre plate and liquid culture medium. The test compounds were added together to each well at a range of concentrations starting at their respective MIC values and descending in doubling dilutions as with the MIC assay. Typically an 8×8 array of wells could be used to combine 8 different concentrations of compound A (from its MIC downwards, including zero) with 8 different concentrations of compound B (ditto).

The wells were inoculated with freshly grown micro-organism and incubated under the conditions described above.

As for the MIC assay, the results were read by the naked eye. A minimum inhibitory concentration was recorded for each combination of A and B. A fractional FIC index (FICI) was then calculated for each compound in that mixture, and these two indices were added together to give an overall FICI indicative of the mode of interaction.

Thus for each mixture tested, the FIC for compound A (FIC_{A})=MIC for (A+B)/MIC for A alone. Similarly the FIC for compound B (FIC_{B})=MIC for (A+B)/MIC for B alone. The overall FICI=FIC_{A}+FIC_{B}.

An FICI of 0.5 or less was taken to indicate synergy, a value greater than 0.5 to 4.0 an indifferent effect and values greater than 4.0 antagonism (i.e. the two compounds counter one another's activity, leading overall to a diminished antimicrobial effect) (see Odds FC, “Synergy, antagonism, and what the chequerboard puts between them”, J Antimicrob Chemother, 2003; 52:1). These results can be depicted visually on a plot (isobologram) of FIC_{A} against FIC_{B} for the mixtures tested.

### Example 1

### Activity Against Propionibacterium spp (Disc Diffusion Assays)

The following experiments all used P. acnes NCTC 737 as the test organism.

MIC, MBC and DDA assays, as described above, were carried out using the test compound t-butyl-p-hydroquinone (TBHQ) and a range of bismuth salts (all ex-Sigma Aldrich, UK).

Each bismuth salt was then subjected, together with the TBHQ, to the SDDA assay described above. In each case, increases in zone diameter (mm) were measured with respect to those observed for the compound showing the larger zone diameters during the previous disc diffusion assays on the individual compounds.

For the (S)DDA experiments, 200 μg of each test compound was loaded onto each disc. The TBHQ was dissolved in ethanol and the bismuth salts in DMSO.

All the (S)DDA experiments were conducted in triplicate.

The MIC and MBC results are shown in Table 1 below and the (S)DDA results in Table 2.

TABLE 1

| | MIC | MBC | MIC/MBC |
|---|---|---|---|
| Test compound | (μg/ml) | (μg/ml) | ratio |
| TBHQ | 7.8 | 7.8 | 1 |
| Bismuth acetate | 0.98 | 7.8 | 0.125 |
| Bismuth chloride | 0.49 | 3.9 | 0.125 |
| Bismuth subsalicylate | 0.98 | 15.6 | 0.06 |
| Bismuth citrate | 0.98 | 7.8 | 0.125 |

TABLE 2

| | | | | SDDA |
|---|---|---|---|---|
| | | SDDA with | SDDA | area |
| | DDA | TBHQ | increase | increase |
| Test compound | (mm) | (mm) | (mm) | (%) |
| TBHQ | 11.04 | | | |
| | (±0.83) | | | |
| Bismuth acetate | 28.17 | 34.89 | 6.72 | 53.4 |
| | (±1.42) | (±0.48) | | |
| Bismuth chloride | 26.07 | 31.64 | 5.57 | 47.29 |
| | (±0.73) | (±0.48) | | |
| Bismuth subsalicylate | 21.02 | 31.85 | 10.83 | 129.59 |
| | (±0.48) | (±0.63) | | |
| Bismuth citrate | 34.47 | 43.41 | 8.94 | 58.59 |
| | (±4.28) | (±1.84) | | |

These data show that TBHQ alone is active against P. acnes NCTC 737, as are the bismuth salts tested.

When the quinone is combined with a bismuth salt, however, the data demonstrate in each case a synergistic antimicrobial interaction between the two, with a significant increase in zone diameter over that exhibited by either compound alone.

A particularly strong synergistic interaction is observed when the quinone is combined with bismuth subsalicylate.

In a separate experiment, the above tests were repeated for TBHQ and bismuth subnitrate (ex-Sigma Aldrich), which was also dissolved in DMSO and loaded at 200 μg per disc for the (S)DDA assays. The results are shown in Tables 3 and 4 below.

TABLE 3

| | MIC | MBC | MIC/MBC |
|---|---|---|---|
| Test compound | (μg/ml) | (μg/ml) | ratio |
| TBHQ | 7.8 | 7.8 | 1 |
| Bismuth subnitrate | 3.9 | 7.8 | 0.5 |

TABLE 4

| | | | | SDDA |
|---|---|---|---|---|
| | | SDDA with | SDDA | area |
| | DDA | TBHQ | increase | increase |
| Test compound | (mm) | (mm) | (mm) | (%) |
| TBHQ | 14.68 | | | |
| | (±0.36) | | | |
| Bismuth subnitrate | 24.91 | 30.39 | 5.48 | 48.84 |
| | (±1.56) | (±0.54) | | |

Again the quinone/bismuth salt combination can be seen to exhibit antimicrobial synergy against P. acnes NCTC 737.

### Example 2

### Activity Against Propionibacterium spp (FIC Assays)

Mixtures of TBHQ and bismuth subsalicylate (BisSS), containing various relative proportions of the two actives, were then subjected to FIC assays against P. acnes NCTC 737, as described above. The results were used to prepare FIC isobolograms. All assays were conducted in triplicate.

The overall FICI obtained for the mixtures was 0.31, representing the mean of three replicates. This indicates a synergistic interaction. A representative isobologram is shown in FIG. 1; the dashed line indicates where overall FICIs (i.e., FIC_{TBHQ}+FI_{BisSS}) equal 1, which would indicate a purely indifferent interaction. FIG. 1 clearly demonstrates the synergistic activity of the combination of the quinone and the bismuth salt against P. acnes NCTC 737.

### Example 3

### Activity Against Propionibacterium spp (Other Quinones)

Four further quinone/bismuth salt combinations according to the invention were tested for activity against P. acnes NCTC 737.

DDA assays, as described above, were carried out using each of the test compounds individually. The quinone/bismuth salt combinations were then subjected to the SDDA assay described above. In each case, increases in zone diameter (mm) were measured with respect to those observed for the compound showing the larger zone diameters during the previous disc diffusion assays on the individual compounds.

For the (S)DDA experiments, 200 μg of each test compound was loaded onto each disc, with the exception of 2-t-butyl-p-benzoquinone which was used at 100 μg per disc. The quinones and bismuth salts were all dissolved in DMSO.

All the (S)DDA experiments were conducted in triplicate.

The results are shown in Table 5 below.

TABLE 5

| | | | | SDDA |
|---|---|---|---|---|
| | | | SDDA | area |
| | DDA | SDDA | increase | increase |
| Test compound/combination | (mm) | (mm) | (mm) | (%) |
| Bismuth subsalicylate (BisSS) | 19.12 | | | |
| | (±0.18) | | | |
| 2-chloro-p-benzoquinone | 25.43 | | | |
| | (±0.54) | | | |
| BisSS + 2-chloro-p-benzoquinone | | 31.73 | 6.30 | 55.69 |
| | | (±1.29) | | |
| Bismuth citrate (BisCit) | 32.97 | | | |
| | (±2.64) | | | |
| 2-t-butyl-p-benzoquinone | 52.61 | | | |
| | (±2.81) | | | |
| BisCit + 2-t-butyl-p-benzoquinone | | 58.71 | 6.10 | 24.53 |
| | | (±0.65) | | |
| Bismuth citrate (BisCit) | 32.97 | | | |
| | (±2.64) | | | |
| Methyl-p-benzoquinone | 39.90 | | | |
| | (±0.95) | | | |
| BisCit + methyl-p-benzoquinone | | 45.99 | 6.09 | 32.86 |
| | | (±2.02) | | |
| Bismuth subnitrate (BisSN) | 24.91 | | | |
| | (±1.56) | | | |
| 2-chloro-p-benzoquinone | 25.43 | | | |
| | (±0.54) | | | |
| BisSN + 2-chloro-p-benzoquinone | | 34.11 | 8.68 | 79.91 |
| | | (±0.54) | | |

In each case, the quinone/bismuth salt combination exhibits antimicrobial synergy, with a significant increase in zone diameter over that exhibited by either compound alone.

### Example 4

### Activity Against Other Propionibacteria

The activities of TBHQ, bismuth subsalicylate (BisSS), bismuth citrate (BisCit) and bismuth subnitrate (BisSN) were assessed against a panel of different propionibacteria using the MIC and MBC assays for TBHQ and the MIC by agar dilution assay for the bismuth salts, as described above. The results are shown in Table 6, which also indicates the resistance phenotype for each of the test strains.

TABLE 6

| | | TBHQ | TBHQ | BisSS | BisCit | BisSN |
|---|---|---|---|---|---|---|
| | Resistance | MIC | MBC | MIC | MIC | MIC |
| Test organism | phenotype | (μg/ml) | (μg/ml) | (μg/ml) | (μg/ml) | (μg/ml) |
| P. granulosum NCTC 11865 | None | 3.9 | 7.8 | 3.9 | 7.8 | 7.8 |
| P. acnes PRP-002 | Tet/MLS | 3.9 | 7.8 | 3.9 | 7.8 | 7.8 |
| P. acnes PRP-003 | Tet | 7.8 | 7.8 | 1.95 | 3.9 | 7.8 |
| P. acnes PRP-004 | Tet | 1.95 | 7.8 | 3.9 | 7.8 | 7.8 |
| P. granulosum PRP-005 | MLSK | 62.5 | 62.5 | 3.9 | 7.8 | 7.8 |
| P. granulosum PRP-006 | MLS | 7.8 | 7.8 | 1.95 | 7.8 | 7.8 |
| P. acnes PRP-007 | Clin | 3.9 | 7.8 | 1.95 | 3.9 | 7.8 |
| P. acnes PRP-008 | Clin | 3.9 | 7.8 | 1.95 | 3.9 | 7.8 |
| P. acnes PRP-010 | MLSK | 3.9 | 15.6 | 3.9 | 7.8 | 7.8 |
| P. acnes PRP-017 | MLS | 3.9 | 7.8 | 1.95 | 3.9 | 7.8 |
| P. granulosum PRP-019 | MLSK | 31.25 | 31.25 | 3.9 | 7.8 | 7.8 |
| P. granulosum PRP-021 | MLS | 15.6 | 31.25 | 3.9 | 7.8 | 7.8 |
| P. acnes PRP-023 | MLSK | 3.9 | 7.8 | 1.95 | 3.9 | 7.8 |
| P. acnes PRP-026 | MLS | 3.9 | 7.8 | 1.95 | 3.9 | 7.8 |
| P. acnes PRP-039 | Tet/MLS | 3.9 | 7.8 | 1.95 | 0.98 | 7.8 |
| P. granulosum PRP-043 | MLS | 15.6 | 15.6 | 1.95 | 3.9 | 7.8 |
| P. granulosum PRP-044 | MLS | 15.6 | 31.25 | 3.9 | 7.8 | 7.8 |
| P. acnes PRP-046 | None | 1.95 | 7.8 | 1.95 | 3.9 | 7.8 |
| P. acnes PRP-053 | Tet/MLS | 3.9 | 7.8 | 3.9 | 3.9 | 7.8 |
| P. granulosum PRP-055 | None | 3.9 | 7.8 | 3.9 | 7.8 | 7.8 |
| P. acnes PRP-059 | MLS | 3.9 | 7.8 | 3.9 | 7.8 | 7.8 |
| P. acnes PRP-068 | Ery | 3.9 | 7.8 | 3.9 | 7.8 | 7.8 |
| P. acnes PRP-101 | Tet/MLS | 3.9 | 7.8 | 3.9 | 7.8 | 7.8 |
| P. acnes PRP-102 | Tet/MLS | 7.8 | 15.6 | 1.95 | 7.8 | 7.8 |

[Abbreviations: American Type Culture Collection (ATCC), National Collection of Type Cultures (NCTC), Propionibacterium Panel Number (PRP), Tetracycline (Tet), Erythromycin (Ery), Clindamycin (Clin), Macrolide-Lincosamide-Streptogramin (MLS), Macroliode-Lincosamide-Streptogramin-Ketolide (MLSK)]

(S)DDA assays were then conducted using the TBHQ and BisSS individually and in combination, against the same panel of test organisms. The solvents used were ethanol for the TBHQ and DMSO for the BisSS, and again 200 μg of each test compound was loaded onto each disc. The results are shown in Table 7.

TABLE 7

| | | | TBHQ + | SDDA | |
|---|---|---|---|---|---|
| | TBHQ | BisSS | BisSS | increase | SDDA area |
| Test organism | DDA (mm) | DDA (mm) | SDDA (mm) | (mm) | increase (%) |
| P. granulosum NCTC 11865 | 11.50 | 16.36 | 23.51 | 7.15 | 106.51 |
| | (±0.93) | (±0.47) | (±0.18) | | |
| P. acnes PRP-002 | 18.12 | 19.99 | 28.06 | 8.07 | 97.04 |
| | (±0.18) | (±1.71) | (±0.95) | | |
| P. acnes PRP-003 | 18.12 | 18.95 | 27.13 | 8.18 | 104.97 |
| | (±0.47) | (±1.891 | (±1.00) | | |
| P. acnes PRP-004 | 19.88 | 17.61 | 26.82 | 6.94 | 82.01 |
| | (±1.08) | (±1.71) | (±0.78) | | |
| P. granulosum PRP-005 | 10.67 | 14.81 | 23.92 | 9.11 | 160.86 |
| | (±0.72) | (±1.00) | (±0.31) | | |
| P. granulosum PRP-006 | 9.42 | 18.43 | 22.99 | 4.56 | 55.61 |
| | (±0.78) | (±0.36) | (±0.82) | | |
| P. acnes PRP-007 | 12.74 | 16.16 | 29.31 | 13.15 | 228.96 |
| | (±0.82) | (±0.31) | (±0.90) | | |
| P. acnes PRP-008 | 17.92 | 19.47 | 28.58 | 9.11 | 115.47 |
| | (±2.00) | (±1.26) | (±1.61) | | |
| P. acnes PRP-010 | 18.54 | 14.39 | 24.85 | 6.31 | 79.65 |
| | (±1.40) | (±0.18) | (±1.73) | | |
| P. acnes PRP-017 | 13.98 | 16.78 | 25.79 | 9.01 | 136.22 |
| | (±0.93) | (±0.54) | (±1.42) | | |
| P. granulosum PRP-019 | 10.36 | 16.78 | 26.72 | 9.94 | 153.56 |
| | (±0.47) | (±0.93) | (±3.77) | | |
| P. granulosum PRP-021 | 0.00 | 19.05 | 26.20 | 7.15 | 89.15 |
| | (±0) | (±2.00) | (±3.17) | | |
| P. acnes PRP-023 | 18.54 | 19.05 | 25.68 | 9.52 | 152.53 |
| | (±1.77) | (±1.53) | (±1.53) | | |
| P. acnes PRP-026 | 13.98 | 16.16 | 25.99 | 7.76 | 103.25 |
| | (±0.82) | (±1.08) | (±2.07) | | |
| P. acnes PRP-039 | 17.71 | 18.23 | 28.79 | 14.29 | 294.23 |
| | (±0.31) | (±0.18) | (±1.09) | | |
| P. granulosum PRP-043 | 0.00 | 14.50 | 26.41 | 9.53 | 144.79 |
| | (±0) | (±0.18) | (±2.24) | | |
| P. granulosum PRP-044 | 0.00 | 16.88 | 24.34 | 8.08 | 124.08 |
| | (±0) | (±1.29) | (±0.90) | | |
| P. acnes PRP-046 | 20.71 | 16.26 | 30.14 | 12.85 | 203.87 |
| | (±0.36) | (±1.26) | (±2.49) | | |
| P. acnes PRP-053 | 17.09 | 17.29 | 25.68 | 7.66 | 103.09 |
| | (±0.82) | (±1.59) | (±0.47) | | |
| P. granulosum PRP-055 | 18.02 | 16.57 | 27.34 | 7.15 | 83.37 |
| | (±0.62) | (±0.95) | (±1.42) | | |
| P. acnes PRP-059 | 19.88 | 20.19 | 31.79 | 12.60 | 174.43 |
| | (±1.89) | (±0.00) | (±2.49) | | |
| P. acnes PRP-068 | 16.78 | 19.99 | 27.34 | 9.11 | 124.92 |
| | (±0.93) | (±1.29) | (±0.54) | | |
| P. acnes PRP-101 | 12.84 | 18.23 | 26.61 | 6.21 | 70.15 |
| | (±1.26) | (±1.40) | (±1.00) | | |
| P. acnes PRP-102 | 14.50 | 20.40 | 28.58 | 8.18 | 96.27 |
| | (±2.25) | (±0.95) | (±1.73) | | |

The data in Tables 6 and 7 show both TBHQ and BisSS to be active against all of the propionibacterial strains tested. Combinations of the two actives, however, appear from the (S)DDA results to be acting synergistically against most of the test organisms, with significant increases in zone diameters and areas. Antibacterial synergy is observed against many of the antibiotic resistant strains, a fact likely to be of considerable clinical importance.

### Example 5

### Topical Anti-Acne Formulations

The results from Examples 1 to 4 show that the combination of a quinone and a bismuth salt can be an effective antimicrobial agent, in particular against the bacteria associated with acne, with a synergistic impact on the antimicrobial activity of the combination compared to those of the individual compounds alone. This can be of use in preparing antimicrobial formulations, in particular for topical application to the skin, for either prophylactic or therapeutic use in any context where such bacteria are thought to be involved as possible sources of infection.

Even in cases where a combination of a quinone and a bismuth salt has an additive, as opposed to synergistic, antimicrobial activity compared to those of the individual compounds, this can be of considerable benefit when preparing formulations for topical use. One of the compounds may be used to replace a proportion of the other, thus lowering any side effects and/or other undesirable properties of the combination without undue loss of antimicrobial activity.

A topical formulation for use in treating acne may for example be prepared by combining a quinone, in particular an alkyl-substituted benzo/hydroquinone such as TBHQ, with a bismuth salt such as bismuth subsalicylate, in a suitable fluid vehicle and optionally together with conventional additives. Such vehicles and additives may be for instance as found in Williams' “Transdermal and Topical Drug Delivery”, Pharmaceutical Press, 2003 and other similar reference books, and/or in Rolland A et al, “Site-specific drug delivery to pilosebaceous structures using polymeric microspheres”, Pharm. Res. 1993; 10: 1738-44; Mordon S et al, “Site-specific methylene blue delivery to pilosebaceous structures using highly porous nylon microspheres: an experimental evaluation”, Lasers Surg. Med. 2003; 33: 119-25; and Alvarez-Roman R et al, “Skin penetration and distribution of polymeric nanoparticles”, J. Controlled Release 2004; 99: 53-62.

The formulation may be prepared and administered using known techniques. It may for example take the form of a cream, lotion or gel.

The concentrations of the two active agents may be in the ranges described above, and will be determined based on the intended use of the formulation, its intended mode of administration and the activities of the particular chosen active agents.

### Example 6

### Activity Against Bacteria Associated with Oral Health (MIC and MBC Assays)

The following experiments all used Por. gingivalis NCTC 11834 as the test organism.

MIC and MBC assays, as described above, were carried out using the test compounds t-butyl-p-hydroquinone (TBHQ) and bismuth subsalicylate (both ex-Sigma Aldrich). The TBHQ was dissolved in ethanol and the bismuth subsalicylate in DMSO. All the experiments were conducted in triplicate.

The results are shown in Table 8 below.

TABLE 8

| | MIC | MBC | MIC/MBC |
|---|---|---|---|
| Test compound | (μg/ml) | (μg/ml) | ratio |
| TBHQ | <0.49 | 0.49 | <1 |
| Bismuth subsalicylate | <0.49 | 1.95 | <0.25 |

These data show that both the TBHQ and the bismuth subsalicylate alone are active against Por. gingivalis NCTC 11834.

### Example 7

### Activity Against Bacteria Associated with Oral Health (FIC Assays)

Mixtures of TBHQ and bismuth subsalicylate (BisSS), containing various relative proportions of the two actives, were then subjected to FIC assays against Por. gingivalis NCTC 11834, as described above. The results were used to prepare FIC isobolograms. All assays were conducted in triplicate.

The overall FICI obtained for the mixtures was 0.5, representing the mean of three replicates. This indicates a synergistic interaction. A representative isobologram is shown in FIG. 2; the dashed line indicates where overall FICIs (i.e., FIC_{TBHQ}+FIC_{BisSS}) equal 1, which would indicate a purely indifferent interaction. FIG. 2 clearly demonstrates the synergistic activity of the combination of the quinone and the bismuth salt against Por. gingivalis NCTC 11834.

### Example 8

### Topical Oral Health Care Formulations

Examples 6 and 7 above indicate the utility of quinone/bismuth salt combinations in treating bacterial infections within the oral cavity, for example dental caries, halitosis, oral thrush and in particular periodontal diseases such as gingivitis and periodontitis. Such combinations may also be used for general oral health care, for example for the creation and/or maintenance of fresh-smelling breath, or in the prevention and/or reduction of plaque formation.

A topical formulation for use in this way, for example against bacteria such as P. gingivalis, may be prepared by combining a quinone, in particular an alkyl-substituted benzo/hydroquinone such as TBHQ, with a bismuth salt such as bismuth subsalicylate, in a suitable fluid vehicle and optionally together with conventional additives, as described above.

The formulation may be prepared and administered using known techniques. It may for example take the form of a paste, cream, gel, lozenge, buccal patch, spray, mouthwash or dentifrice. It may be carried in or on a dental fibre or tape. It may contain additives which target the active ingredients to a particular site within the oral cavity, for example the gums or teeth, and/or which otherwise control the release of the actives at the relevant site.

1. An antimicrobial formulation comprising a quinone, wherein the formulation comprises a compound or mixture of compounds selected from the group consisting of: (i) a benzoquinone, (ii) a hydroquinone, (iii) a mixture of benzoquinone and hydroquinone, and (iv) a bismuth salt.

2. (canceled)

3. A formulation according to claim 1, which is suitable for topical application to the skin.

4. (canceled)

5. A formulation according to claim 1, wherein the two C═O groups or C—OH groups of the benzoquinone or hydroquinone are positioned para to one another.

6. A formulation according to claim 1, wherein the quinone is an alkyl-substituted benzoquinone, an alkyl-substituted hydroquinone or a mixture thereof.

7-9. (canceled)

10. A formulation according to claim 1 wherein the quinone is t-butyl hydroquinone (TBHQ), 2-t-butyl-p-benzoquinone (TBBQ) or a mixture thereof.

11. (canceled)

12. A formulation according to claim 1 wherein the bismuth salt is selected from bismuth carboxylates, bismuth halides, bismuth sulphadiazine, bismuth sulphate, bismuth nitrate, bismuth subnitrate, bismuth subgallate, bismuth oxide, bismuth oxychloride, bismuth hydroxide, bismuth phosphate, bismuth aluminate, bismuth tribromophenate, bismuth thiol, bismuth peptides, bismuth salts of quinolines and their derivatives (eg, bismuth hydroxyquinolines), bismuth pyrithione and other bismuth salts of pyridine thiols, and mixtures thereof.

13-26. (canceled)

27. A kit for preparing an antimicrobial formulation, the kit comprising a source of a quinone and a source of a bismuth salt, together with instructions for combining the two compounds so as to make the formulation at or before the point of its intended application, and/or for the co-administration of the two compounds.

28-37. (canceled)

38. A method of treatment of a human or animal patient suffering from or at risk of suffering from a condition which is caused by, transmitted by and/or exacerbated by microbial, in particular bacterial, activity, the method involving administering to the patient a therapeutically or prophylactically effective amount of an antimicrobial formulation containing a quinone and a bismuth salt.

39. A method according to claim 38, wherein the condition comprises acne and/or acne lesions.

40. A method according to claim 38, wherein the condition is a condition within the oral cavity.

41-43. (canceled)

44. A formulation according to claim 1, wherein the quinone is substituted with one or more groups selected from alkyl groups; alkoxyl groups; halogens; nitro groups —NO₂; and amino groups —NR₂ wherein each R is independently either hydrogen or C₁ to C₆ alkyl.
